(19) United States
(12) Patent Application Publication
(10) Pub. No.: US 2009/0269405 A1
(43) Pub. Date: Oct. 29, 2009

(54) ENZYME MEDIATED DELIVERY SYSTEM

(75) Inventors: J. Brian Windsor, Austin, TX (US); Nitin Nitin, Helotes, TX (US)

Correspondence Address: CHALKER FLORES, LLP, 2711 LBJ FRWY, Suite 1036, DALLAS, TX, 75234, US

(73) Assignee: Appian Labs, LLC, Austin, TX (US)

(21) Appl. No.: 12/420,745

(22) Filed: Apr. 8, 2009

Related U.S. Application Data
(60) Provisional application No. 61/043,386, filed on Apr. 8, 2008.

(51) Int. Cl. A61K 9/14

(52) U.S. Cl. 424/484

(57) ABSTRACT

The present invention includes compositions, methods, and systems for the development of a novel delivery vehicle that affects release of an agent upon the degradation of components of said vehicle by one or more enzymes. In one example, the system comprises components designed to degrade upon the presence of desired concentrations of proteinases, specifically matrix metalloproteinases, and subsequent release of the agent.

### CROSS-REFERENCE TO RELATED APPLICATIONS

This application claims priority to U.S. Provisional Application Ser. No. 61/043,386, filed Apr. 8, 2008, the entire contents of which are incorporated herein by reference.

### TECHNICAL FIELD OF THE INVENTION

The present invention relates in general to the field of the drug delivery, and more particularly, to novel compositions and methods for enzyme-mediated delivery of therapeutics.

### STATEMENT OF FEDERALLY FUNDED RESEARCH

None.

### BACKGROUND OF THE INVENTION

Without limiting the scope of the invention, this invention relates generally to the field of drug delivery, and, more particularly, to the development of an enzyme-mediated drug delivery system. A wide range of existing and near-term therapeutics have great potential, but many possess drawbacks in the delivery system that slow or prevent implementation for aiding human health. Fortunately, the physical, chemical, and/or biological nature of a promising drug candidate may sometimes be assisted by modifying the delivery system of the drug.

### SUMMARY OF THE INVENTION

While several developments exist in the drug delivery field, including sustained and controlled release applications, methods and compositions are needed for targeted delivery of drugs using vehicles broken down by enzymes. The present invention allows for the delivery of a drug or active agent payload in an enzyme-mediated and enzyme concentration dependent manner. For example, the delivery system of the present invention will release its payload upon the targeting milieu reaching a threshold enzyme concentration. Unlike current systems that depend on enzymatic processing of a pro-drug, the present invention delivers the full payload of the drug or active agent when the releasing enzyme, e.g., a protease, breaks through the exterior coating of the drug delivery vehicle and/or the breakdown of the matrix or polymer in which the drug or active agent is embedded and/or in which it is coated. Presently, none of the sustained and controlled released applications and methods have used novel polymer or matrix for proteases as described herein.

In one embodiment, the present invention includes compositions and methods of controlling the release of an active agent with a proteinase comprising: encapsulating one or more active agents with a crosslinked or uncrosslinked matrix, the matrix being cleavable by the proteinase; wherein exposure of the matrix to one or more proteinases causes the cleavage of the matrix and the release of the active agents. In one aspect, the matrix is formulated such that cleavage and degradation of the matrix occurs in a proteinase enzyme concentration specific manner. In another aspect, the concentration of proteinase comprises a threshold concentration below which agents present within the matrix are not released. In another aspect, the threshold concentration comprises a level that is higher than a basal level of proteinase enzyme activity in normal tissue. In another aspect, the threshold concentration comprises a level that is higher than a basal level of proteinase enzyme activity in normal tissue over a specific time period. In another aspect, the proteinase is a matrix metalloproteinase (MMP).

In one embodiment, the present invention includes a tunable system for delivery of agents comprising a matrix comprising an active agent and a substrate for a proteinase enzyme and which, upon exposure to the proteinase is degraded thereby triggering the release of the active agent. In one aspect, the matrix is crosslinked. In another aspect, the time and rate of delivery is tuned by adjusting the amount of substrate, the amount of crosslinking, the type of crosslinking and combinations thereof.

The present invention includes a novel delivery vehicle that affects the release of an agent upon enzymatic degradation of components of the vehicle by one or more enzymes, e.g., proteases, nucleases, glycosylases, lipidases and combinations thereof. The balance between degradation and synthesis of the extracellular matrix (ECM) is carefully regulated; therefore, significant alterations in matrix turnover lead to a wide range of pathological conditions especially because so many developmental processes, such as embryonic development, morphogenesis, cellular reproduction and tissue growth, are dependent on ECM degradation.

More particularly, the present invention includes compositions and methods for encapsulating or controlling the release of one or more active agents. The composition and method of making a composition that controls the release of an active agent with a metalloproteinase includes encapsulating one or more active agents with a polymer or polymer-polypeptide matrix, wherein the polymer may or may not be crosslinked, including crosslinking by polypeptides but which, by virtue of the composition of the polymer and/or crosslinking agent or polypeptide are susceptible to cleavage by metalloproteinases; and exposing the matrix to one or more metalloproteinases, wherein the proteolytic activity of the metalloprotease(s) upon the peptides within, e.g., a polymer or matrix to which the peptide is attached or bound causes the release of the encapsulated active agents. In one example, the composition comprises a biopolymer. The polymer or polypeptides may comprise at least one of collagen (Types I to XIV), elastin, gelatin, fibronectin, laminin and basal membrane proteins. The composition may be made into a thin film adapted for, among others topical, parenteral, oral, buccal or subcutaneous administration. The composition may be formulated into a pharmaceutical, cosmetic, cosmoceutical, use. The composition may be formulated as nano, micro or mini capsules. The composition may be formulated as nano, micro or mini capsules and are combined into a multi-release formulation. The composition may be a hydrogel. The composition may be used to coat a device or integrated into a delivery vehicle.

In one aspect, the composition is incorporated into an implant, a bandage or a dressing. In another aspect, the composition is formed into multiple particles with different drugs and the matrix is a substrate for the same metalloproteinases. In another example, the composition is formed into multiple particles with different drugs and the polypeptides are a substrate for different metalloproteinases. The composition may even comprise multiple layers that are susceptible to a first and a second metalloproteinases or even multiple layers each loaded with a different active agent and wherein each layer is susceptible to different metalloproteinases. The composition matrix may comprise dyes, tracers, labels, contrast agents, or other detection agents. For example, the composition may be formulated or loaded into a plurality of beads, each bead comprising a different polymer-polypeptide matrix matched with a particular detection agent, where upon exposure to a delivery site, the presence or absence of a metalloprotease at the delivery site is detected by the detection agent released from the beads. In another example, the composition comprises at least a portion of an indicator, wherein the indicator is placed in a wound, derma, cosmetic/cosmoceutical, oral, implants, tumors (metastasis) or a device and the active agent is an indicator of release. In yet another example, the composition is formulated into a plurality of beads, each bead comprising a different polymer-polypeptide matrix matched with a particular detection agent, whereupon exposure to a delivery site, the presence or absence of a metalloproteinases at the delivery site is detected by the detection agent released from the beads on a cardiac device. In one aspect, the polymer-polypeptide matrix further comprises one or more tissue inhibitors of metalloproteinases.

In another embodiment the compositions and methods of the present invention include a composition for the controlled release of an active agent comprising: a polymer-polypeptide matrix comprising a polymeric portion crosslinked by a protein that is susceptible to cleavage by a metalloproteinase, wherein erosion of the polymer-polypeptide by exposure to a metalloproteinase that is specific for the protein causes the release of one or more active agents that are restrained by the polymer-polypeptide matrix.

In yet another aspect of the present invention, the composition for the controlled release of an active agent is made by a method comprising: encapsulating one or more active agents with a polymer-polypeptide matrix, wherein the polypeptides are crosslinked to the polymer and are susceptible to cleavage by metalloproteinases; and exposing the polymer-polypeptide matrix to one or more metalloproteinases, wherein the cleavage of the polypeptide causes the release of the encapsulated active agents. In another aspect, the matrix comprises a gelatin coacervate. In one aspect, the matrix comprises a protein microparticle, a small molecular weight surfactant, one or more active agents and a protein crosslinker. In another aspect, the matrix comprises a collagen microparticle, sodium cholate, one or more active agents and a collagen crosslinker or microparticles that further comprise at least one of a dye, an excipient, a stabilizer, a buffering agent, an anti-oxidant, a salt, or one or more inert agents. In one aspect, the proteinase is a matrix metalloproteinase 1, MMP2, MMP8, MMP9 and Gelatinase. In another embodiment, the present invention includes a polymer-polypeptide matrix for the controlled release of an active agent made by a method comprising: encapsulating one or more active agents with a polymer-polypeptide matrix, wherein the polypeptide are susceptible to cleavage by metalloproteinases; and exposing the polymer-polypeptide matrix to one or more metalloproteinases, wherein the cleavage of the polypeptide causes the release of the encapsulated active agents. In one aspect, the polymer-polypeptide matrix further comprises one or more protease inhibitors.

### BRIEF DESCRIPTION OF THE DRAWINGS

For a more complete understanding of the features and advantages of the present invention, reference is now made to the detailed description of the invention along with the accompanying figures and in which:

FIG. 1 shows the release of fluorescence dye from PLGA microspheres in a gelatin hydrogel 75:25 (bloom 300) treated with MMP-9 enzymes at 37° C.;

FIG. 2 shows the release of fluorescence dye from PLGA microspheres in a gelatin hydrogel 50:50 (bloom 300) treated with MMP-9 enzymes at 37° C.;

FIG. 3 shows the degradation of Gelatin shell in microcapsules treated with varying concentration of MMP-9 enzymes for 12 hours at 37° C. Degradation of shells leads to release of oil phase, which shows aggregation in aqueous buffer; and

FIG. 4 shows the release of fluorescence dye after treatment of gelatin microcapsules with varying concentration of MMP-9 enzymes. The results show ˜15 fold release over control with enzyme concentration of 1 ug/ml.

FIGS. 5A and 5B are SEM images of collagen microparticles.

FIG. 6 shows the imaging distribution of collagen in microparticles.

FIG. 7 is a graph that shows a DSC analysis of stability of collagen in microparticles.

FIG. 8 is a graph that shows the concentration dependent enzyme triggered release of vancomycin from collagen microparticles.

FIG. 9 is a graph that shows the relative measurement of oxygen diffusion as a function of storage conditions in dry capsules in air.

FIG. 10 is a graph that shows the relative measurement of oxygen diffusion as a function of storage conditions in dry capsules in Argon.

FIG. 11 is a graph that shows the relative measurement of oxygen diffusion as a function of storage conditions under aqueous conditions in Argon.

FIGS. 12A and 12B show control the kinetics of release of bioactive compounds with a thin shell (12A) and a thick shell (12B).

FIG. 13 shows the release kinetics of thin vs. thick shell.

FIG. 14 is a graph that compares the release kinetics of thin vs. thick shell wherein the engineered shell thickness reduces the rate of release from core-shell microparticles by ˜3 fold.

FIG. 15 shows the degradation of the shell using activated PBS-buffer 20 minutes after UV exposure, the particles were incubated overnight with the activated PBS sample.

FIG. 16 shows the specificity of the protease against the shell, in this example, a lack of MMP-1 activity after 24 hours to gelatin microparticles which are MMP-9 responsive.

FIG. 17 shows the specificity of the protease against the shell, in this example, a lack of MMP-1 activity after one week to gelatin microparticles which are MMP-9 responsive.

FIG. 18 shows microparticles treated with UV treated 3-D tissue media.

FIG. 19 shows gelatin microcapsules encapsulating benzoyl peroxide.

FIG. 20 shows the encapsulation (top) and release (bottom) of Trypan Blue dye in collagen.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

In one embodiment, the vehicle would be a microcapsule wherein the shell of the capsule contained components subject to degradation by proteinases or other enzymes so that the presence of a certain concentration of these enzymes would cause degradation of the components, associated disruption of the integrity of the microcapsule, and release of the contents of the capsule.

The present invention may include proteins or peptides that are cleaved by a wide variety of proteases (also referred to as proteinases or peptidases), including but not limited to Serine-type peptidases, Threonine-type peptidases, Cysteine-type peptidases, Aspartic-type peptidases, Glutamic-type peptidases, Metallopeptidases, Omega-peptidases, tripeptidyl-peptidases, peptidyl-dipeptidases, dipeptidases, HEXXH motif and non-HEEX motif peptidases, Exopeptidases, Endopeptidases, aminopeptidases, carboxypeptidases and other peptidases that have not been assigned to one of these groups. Depending on the type of peptidase releasing enzyme, the skilled artisan can access one or more peptidase databases, such as MEROPS or PMAP-CutDB, type in the proposed target substrate sequence or proteinases and then select the amino acid sequence for the peptide to include in the matrix or polymer used to coat, encapsulate or intermix with the active agent or drug. The present invention may also include one or more peptidase inhibitors in which the matrix or polymer includes protease inhibitors (also included in the MEROPS or PMAP-CutDB databases) that are used to stop certain endogenous or newly expressed proteases from degrading the compositions of the present invention.

Non-limiting examples of proteases that may be used to trigger the delivery of the payload(s) of the present invention include, Chymotrypsin/trypsin; Lysyl endopeptidase; Streptogrisin A; Dipeptidyl-peptidase 7; Prolyl oligopeptidase; Dipeptidyl-peptidase IV; Acylaminoacyl-peptidase; Glutamyl endopeptidase; Carboxypeptidase C; Lysosomal Pro-X carboxypeptidase; Prolyl aminopeptidase; Endopeptidase IV (sppA); Lactoferrin; Papain; Bleomycin hydrolase; Calpain; Ubiquitin C-terminal hydrolase family 1; Ubiquitin C-terminal hydrolase family 2; Caspases (ICE); Pepsin; Human endopeptidases; Type IV-prepilin leader peptidase; Membrane alanyl aminopeptidase; Peptidyl-dipeptidase A; Thimet oligopeptidase; Collagenases; Matrix metallopeptidases; Dipeptidyl-peptidase III; Carboxypeptidase A; Carboxypeptidase E; Gamma-D-glutamyl peptidase; Leucyl aminopeptidase; Methionyl aminopeptidase, type 1; Aminopeptidase P; Metalloprotease ARX1; Glutamate carboxypeptidase, Peptidase T; Xaa-His dipeptidase; Carboxypeptidase Ss1; Aminopeptidase S; Glutamate carboxypeptidase II; Aminopeptidase T; Dipeptidyl-peptidase III. Generally, the peptides used with the present invention will include or more cleavage sites for these proteases. When using combinations of peptides these are selected such that the release of the active agent or drug is triggered in the presence of certain threshold levels of two or more proteases.

In one embodiment the enzymes would be matrix metalloproteinases (MMPs) and the capsule components would include substrates of these MMPs such as collagen (Types I, II, IV, VII, and others), elastin, gelatin, basal membrane components, or other substrates. In such embodiment the presence of a threshold level of MMP (one or more members of the MMP class, and this could be a tailored feature of the present invention) would cause degradation of the substrate within the shell of the microcapsule to the degree that the integrity of the capsule failed and allowed for release of an agent. Such agent could be a therapeutic (natural or man-made) or cosmetic or other agent. Agents might include retinol, vitamin C, NSAIDs, MMP inhibitors, or other agents. For example, an encapsulation vesicle that uses collagen as a component in a concentration that would be degraded by a certain level of one or more MMPs.

Matrix metalloproteinases (MMPs) are a family of proteolytic enzymes responsible for degradation of the ECM. MMPs are regulated at three different levels: gene transcription, pro-enzyme processing and proteolytic inhibition. It is not surprising that MMP activity is implicated in numerous diseases; nonetheless, there numerous targets at which therapeutic intervention might be directed. (Doherty, et al. 2002).

Twenty-three human MMPs have been identified to date. MMPs exist in either a soluble or membrane-bound form. All MMPs are initially translated as zymogens (pro-MMPs), which are then processed into a catalytically active protein. Some MMPs are synthesized in the membrane-bound form then cleaved into a diffusable molecule. MMPs are often categorized into groups based on substrate specificity, sequence similarity, and domain organization. (Visse and Nagase 2003) (Doherty, et al. 2002). Some MMPs have greater substrate specificity than others (discussed below). MMPs are inhibited by various natural inhibitors and small molecules developed to alter MMP activity.

The first group of soluble MMPs are called collagenases (MMP-1, MMP-8, and MMP-13), which can cleave interstitial collagens I, II, and III at a specific site as well as degrade other ECM and non-ECM molecules. Specifically, MMP-1 is known to degrade collagen I, collagen II, collagen III, gelatin, and proteoglycans. MMP-8 is known to degrade collagens I, II, III, V, VII, IX, and gelatin. MMP-13 in known to degrade collagens I, II, III, IV, IX, X, XIV, fibronectin, and gelatin. Collagenase 4 (MMP-18) is a Xenopus protein. (Doherty, et al. 2002) (Visse and Nagase 2003).

Gelatinases are another group of soluble MMPs, including gelatinase A (MMP-2) and gelatinase B (MMP-9). These proteinases digest collagen and gelatin substrates. Specifically, MMP-2 has been shown to degrade collagens I, II, IV, V, VII, X, XI, XIV, elastin, fibronectin, gelatin, and nidogen. MMP-9 has been shown to degrade collagens I, III, IV, V, VII, X, XIV, elastin, fibronectin, gelatin, and nidogen. (Doherty, et al. 2002) (Visse and Nagase 2003).

Stromelysins are another category of soluble MMPs. This group includes stromelysin 1 (MMP-3) and stromelysin 2 (MMP-10). MMP-3 has been shown to degrade collagens III, IV, V, IX, X, laminin, nidogen, proteoglycans, α2-antiplasmin, and fibronectin. MMP-10 has been shown to degrade collagens III, IV, V, elastin, fibronectin, and gelatin. MMP-3 generally has a proteolytic efficiency higher than that of MMP-10 even though both enzymes have similar substrate specificities. MMP-3 also activates a number of proMMPs. For example, fully active MMP-1 requires the processing of pro-MMP-1 by MMP-3. Stromelysin 3 (MMP-11) degrades serine protease inhibitors (serpins), but is also grouped with “other MMPs” because the sequence and substrate specificity are not consistent with those of MMP-3. (Doherty, et al. 2002) (Visse and Nagase 2003).

Matrilysins are another group of soluble MMPs that includes matrilysin 1 (MMP-7) and matrilysin 2/endometase (MMP-26). Specifically, MMP-7 is known to degrade collagen IV, elastin, fibronectin, gelatin, and laminin. Furthermore, MMP-7 processes cell surface molecules such as pro-α-defensins (cryptdins), Fas-ligand (FasL), pro-tumor necrosis factor (TNF)-α, and E-cadherin. MMP-26 is known to degrade denatured collagen, fibrinogen, fibronectin, and vitronectin. (Doherty, et al. 2002) (Visse and Nagase 2003).

Another group of MMPs is a subfamily of six proteases called membrane type-MMPs (MT1-MMP to MT6-MMP). Four are type I transmembrane proteins (MMP-14, MMP-15, MMP-16, and MMP-24), and two are glycosylphosphatidylinositol (GPI) anchored proteins (MMP-17 and MMP-25). All MT-MMPs can activate proMMP-2 (with the exception of MT4-MMP) as well as digest a number of ECM molecules. Specifically, MMP-14 (MT1-MMP) is known to degrade MMP-2, collagens I, II, III, fibronectin, gelatin, and laminin. MMP-15 (MT2-MMP) is known to degrade MMP-2, collagens I, II, III, fibronectin, laminin and nidogen. MMP-16 (MT3-MMP) is known to degrade MMP-2, collagen I, collagen III, and fibronectin. MMP-24 (MT5-MMP) is known to degrade MMP-2, gelatin, fibronectin, chondroitin, dermitin, and sulfate proteoglycans. MMP-17 (MT4-MMP) is known to degrade fibrin(ogen) and TNF-α. MMP-25 (MT6-MMP or leukolysin) is known to degrade MMP-2, gelatin, collagen IV, and fibronectin. (Doherty, et al. 2002) (Visse and Nagase 2003).

MMP-23 is a novel Type II transmembrane MMP that is also called cysteine array MMP or CA-MMP. This protein is mainly expressed in reproductive tissues and is intracellularly associated with the ER-Golgi complex, while the active enzyme is released into the extracellular matrix. MMP-23 is known to degrade gelatin. This novel MMP has also been grouped with “other MMPs” below. (Doherty, et al. 2002) (Visse and Nagase 2003)

The other MMPs include proteinases that are not classified in the above categories: MMP-12, MMP-19, MMP-20, MMP-22, MMP-27, and MMP-28. MMP-12 is also called metalloelastase or macrophage elastase, wherein it is essential for macrophage migration and mainly expressed in macrophages. MMP-12 is known to degrade elastin, fibronectin and laminin. MMP-19 is also known as RASI-1. MMP-19 degrades MMP-9, gelatin, laminin-1, collagen IV, and fibronectin. MMP-20 is also known as enamelysin, which digests amelogenin found in newly formed tooth enamel. MMP-22 is cloned from chicken fibroblasts, and a human homologue was then identified using EST sequences. MMP-27 is designated as human MMP-22; however, the function of this enzyme is not known. MMP-28 is also called epilysin, which is mainly expressed in keratinocytes and is known to degrade casein. Furthermore, it has been suggested that MMP-28 functions in tissue hemostasis and wound repair based on expression patterns in intact and damaged skin. (Doherty, et al. 2002) (Visse and Nagase 2003).

Control of MMP Activity. Control of ECM degradation is maintained by inhibiting MMP proteolytic activity with proteins called tissue inhibitors of metalloproteinases (TIMPs). Four TIMPs (TIMP-1, TIMP-2, TIMP-3, and TIMP-4) have been identified in vertebrates. TIMPs are endogenous low molecular weight proteins (21-28.5 kDa) that form non-covalent enzyme-inhibitor complexes with active MMPs. TIMPs bind MMPs at a 1:1 ratio under normal physiological conditions. Because changes in TIMP levels affect directly the level of MMP activity, amounts of both MMPs and TIMPs are relevant under pathological conditions. An example of the highly regulated system is obvious in collagen metabolism. A slow collagen turnover rate is not due to an intrinsically low Kₘ of MMPs, but rather due to the precisely regulated activity between MMPs and TIMPs. (Doherty, et al. 2002) (Visse and Nagase 2003) (Peterson 2004).

TIMPs are not only inhibitors of catalytically active MMPs, but are also involved in regulating MMP activity at the level of transcription as well as participating in the activation of the precursor zymogens. For example, TIMPs bind MMP-1, MMP-2, MMP-3 and MMP-9 intermediates in the MMP activation sequence of latent proMMP to an active protease. Furthermore, fibrinolysin or activated plasminogen (plasmin) can activate pro-MMPs through many circuitries, which perhaps indicates that MMP inhibitors block specific arms of this network of interactions. Clearly recognizing the role each biomarker plays will greatly enhance the chance of successful drug development and treatment strategies for disease. (Doherty, et al. 2002) (Visse and Nagase 2003) (Hu, et al. 2007).

MMP and TIMP Related Conditions. Because MMPs are also involved in many physiological processes (embryo implantation, bone remodeling and organogenesis) and have additional roles in the reorganization of tissues during pathological conditions (inflammation, wound healing and invasion of cancer cells), there are a wide variety of diseases and chronic disorders associated with an imbalance in TIMP activity, MMP activity, or a failure of the regulatory mechanisms. TIMPs are implicated in angiogenesis and neovascularization fundamental to wound healing, tumor growth, metastasis, and collateral blood vessel growth related to chronic ischemia. Processes such as ovulation, trophoblast invasion, skeletal and appendageal development, and mammary gland involution rely on the critical regulation of MMP activity. A loss in regulatory control can lead to diseases such as arthritis, cancer, atherosclerosis, heart failure, aneurysms, nephritis, tissue ulcers, fibrosis, osteoporosis, and periodontal disease. Researchers have also demonstrated that regulatory imbalance is associated with cartilage and bone destruction in rheumatoid and osteoarthritis; degradation of myelin-basic protein in neuroinflammatory diseases; opening of the blood-brain barrier following brain injury; and tissue degradation in gastric ulceration. (Whittaker, et al. 1999) (Doherty, et al. 2002) (Visse and Nagase 2003) (Peterson 2004) (Hu, et al. 2007).

MMP Inhibitors. Natural TIMPs with MMP affinity in the picomolar range look like ideal inhibitors for potential therapeutics but they lack fine selectivity. Research efforts have unveiled a wide variety of compounds that inhibit MMP activity. These synthetic MMP inhibitors (MMPIs) have vast potential for therapeutic value, although it is critical that specificity and potential side-effects are assessed. Understanding structural components has improved specificity of MMP/Inhibitor interactions, although it is not really indicative of clinical viability. To date, TIMPs and MMPIs have had variable and unexpected effects in the treatment of different diseases because there are many substrates to which these molecules can bind. For instance, TIMPs and MMPIs have been shown to inhibit a diverse array of other proteases and interact with specific ECM components. For example, TIMP-1 and TIMP-3 also inhibit enzymes associate with inflammatory processes, namely TNF-α converting enzyme (TACE; also known as a disintegrin and metalloproteinase [ADAM]-17) and ADAM-10. Broad inhibition of these proteases has resulted in unacceptable side effects underlying some of the clinical disappointments that occurred when promising MMPIs were evaluated in clinical trials. (Doherty, et al. 2002) (Visse and Nagase 2003) (Hu, et al. 2007).

Many MMPIs were developed as drugs and despite great potential these drugs were not successful or were cancelled during the clinical trials. Some of the these drugs are: Neovastat, a collagenase inhibitor, gelatinase inhibitor and VEGF Receptor-2 antagonist, developed by Aeterna Laboratories to treat cancer or psoriasis; Dermostat, a collagenase inhibitor, developed by CollaGenex Pharmaceuticals Inc. to treat acne; CPA-926, a MMPI, developed by Kureha Chemical Sankyo to treat arthritis; DPC-333, a MMPI and TNF convertase inhibitor, developed by Bristol Myers Squibb to treat arthritis; Rebimastat, a gelatinase inhibitor, developed by Celltech Bristol Myers Squibb to treat cancer. (Peterson 2004).

Tetracylcines have been chemically modified for use as MMPIs. It appears that these tetracyclines reduce MMP activity at the level of expression or perhaps by weakly binding MMPs in vivo. However, the clinical efficacy of treating cardiovascular disease (e.g., atherosclerosis, aneurysm, or heart failure) with tetracyclines has yet to be validated. (Peterson 2004) Monoclonal-antibody derivatives have potential use as drugs because high MMP specificity can be achieved; however, there are technical difficulties with the biotechnological production of macromolecular proteins and patient compliance is unreliable because parenteral administration is required. (Hu, et al. 2007) A number of compounds show a preference for MMPs with a deep S1′ pocket rather than a short S1′ pocket, but selective inhibitors seem to be less effective than broad spectrum inhibitors in animal models of cancer. (Whittaker, et al. 1999).

Problems with MMPIs. Turning off MMP activity is not a viable solution; it must be precisely regulated at basal levels required for normal activity. Detrimental side-effects were apparent in null gene animal models used by researches in the process of validating MMPI efficacy. For example, MMP-9 knockout mice become defective in the remodeling of extracellular matrix (particularly fibrin) and re-epithelization rates increased. (Kilpadi, et al. 2006). From the many studies conducted in animal models, it became apparent that MMP exposure must be controlled in a way that valid comparisons could be made between the pharmacology of broad spectrum and selective MMP inhibitors. (Whittaker, et al. 1999).

One of the most prevalent problems in developing MMPIs as effective drugs is that some compounds are more selective than others. MMPIs can be too broad spectrum lacking selectivity or cause the combined inhibition of several critical MMPs. A drug that blocks an MMP family responsible for normal cell function might counterbalance the beneficial effects of target inhibition. Furthermore, it is possible that MMPIs lose their selectivity at high exposure levels becoming broad spectrum inhibitors. As to date, only one MMPI is on the market (Periostat®). However, despite repeated failures of clinical trials, there has been a continual investment in MMPI development because there is such a vast potential market for a new drug class that offers life-long treatment of rheumatoid or osteoarthritis, metastatic tumor growth and neoplasia, osteoporosis, periodontal disease, aneurysm, heart failure and/or atherosclerosis. (Whittaker, et al. 1999)

One of the challenges in designing MMPIs is determining which MMPs are involved in normal tissue and cellular function (anti-targets) in order to avoid undesirable side-effects or increased patient mortality. Even for drugs with a seemingly likelihood of success in short-term clinical trials, the negative effects of MMPIs were revealed when large populations were studied for long-term. (Peterson 2004) (Overall and Kleifeld 2006).

“Anti-targets” are defined by Overall and Kleifeld as “molecule[s] with essential roles in normal cell and tissue function. Downmodulation of an anti-target results in clinically unacceptable side effects, initiation of disease, or deleterious alterations in disease progression. This results in shorter onset time of the disease, increased disease burden, poorer patient outcome or decreased survival time.” (Overall and Kleifeld 2006).

The drug tanomastat is an example of an inhibitor that probably interacts with MMP anti-targets. Even though tanomastat blocks MMP2, MMP3 and MMP9 with higher specificity than other MMPs, the drug caused side-effects resulting in a clinical outcome worse in patients using tanomastat than those given standard treatment. An example of a substrate that might be a MMP anti-target is connective tissue growth factor (CTGF), which may be cleaved and inactivated by MMP14. Researchers have detected increased levels of CTGF expressed in osteolytic breast carcinoma metastases in bone. Furthermore, the CTGF gene was also functionally validated in a bone metastasis signature expressed by human breast cancer cells. (Overall and Kleifeld 2006)

In early stages, side-effects such as peritoneal irritation and poor tolerability ceased development of potential drugs. For example, batimastat, a drug developed to reduce thickening of the carotid artery after angioplasty, caused unexpectedly high and sustained plasma concentrations after intraperitoneal administration (100-200 ng/mL batimastat was still detectable 28 days after a single dose). GM6001 is another MMPI intended to reduce vessel thickening and collagen deposition in the vessel wall that was cancelled because of these side-effects. (Whittaker, et al. 1999)

Another problematic trend is that constitutive treatments with MMPIs have very deleterious effects over a longer period of time, including onset of the musculoskeletal syndrome (MSS) in humans. MSS is a tendonitis-like fibromylagia or musculoskeletal syndrome that affects the joints in hands, shoulders, arms, and legs. Drug trials for various diseases using MMPIs such as batimastat, marimastat, CGS-27023A and prinomastat resulted in patients developing MSS. Unfortunately, plasma drug concentrations necessary for effective treatment were above the dose toleration limits. Although MSS is dose and time related, it is reversible if the dose is reduced or discontinued as described in more detail below. Nonetheless, not being able to achieve effective dose levels may be one of the reasons that the cancer trials were not successful. Specific MMPIs will be discussed in the next section. (Peterson 2004)

Marimastat is a drug developed to treat arthritis among other conditions. During a clinical study, the drug induced MSS in humans when pateints were exposed to long-term dosing. Symptoms were joint pain, stiffness, edema, skin discoloration, and restriction of movement. This included inflammation and tenderness in the small joints of the hand and in the shoulder girdle, then moved to other joints in the arms and legs if dosing continued unchanged. The symptoms were not only dose related but progressed over time in 10 of the 30 patients. Onset of musculoskeletal toxicity for five of the patients with severe events varied from 56 days (75 mg twice daily) to 199 days (25 mg daily). It appears that the upper dose limit after 1 month of treatment was no more than 50 mg twice daily. Nonsteroidal anti-inflammatory agents, analgesics and NSAIDs did not alleviate symptoms, but the condition was reversible in some cases. Some patients could continue treatment after a 2-4 week drug holiday. (Peterson 2004) (Whittaker, et al. 1999)

In other clinical trials, adverse side effects were not apparent when marimastat was combined with other MMPIs. For example, marimastat was combined with carboplatin in a clinical trial to treat patients with advanced ovarian cancer. Neither drug induced side-effects when two agents were administered in combination. Similarly, patients with pancreatic cancer seem to tolerate the combination of marimastat and gemcitabine. Furthermore, it is apparent from animal cancer models that treatment with MMPIs will be most effective when used in combination with chemotherapy at an early stage of the cancer. (Whittaker, et al. 1999).

Periostat® is the only known MMPI on the market. The drug was launched by the company CollaGenex Pharmaceuticals Inc. to treat the disease periodontitis. Also known as doxycycline, it is a tetracycline analog which lacks anti-bacterial activity. It is known to inhibit collagenase (MMP-1) activity; however, it is not entirely clear whether by direct inhibition of MMP activity or indirect decrease in collagenase expression. Side-effects were noticeable after long-term treatment. In fact, the Periostat® label lists diarrhea, heartburn, joint pain, and nausea as some of the possible side-effects. In a phase II clinical study for chronic oral doxycycline treatment (100 mg bid), five out of 33 patients developed abdominal aortic aneurysms. Another study shows that most patients do not actually take the medication as directed (90% patients receiving doxycycline reported taking their medication as directed but 16% actually managed this level of compliance). Perhaps this explains why Periostat® is not the blockbuster drug anticipated. (Peterson 2004).

The Need for Normal MMP Activity. Maintenance of normal MMP activity is critical, which is readily apparent in wound care. MMPs are responsible for ECM degradation; break down of growth factors and growth factor receptors; as well as activation of latent growth factors. During tissue injury, the damaged matrix proteins are degraded and removed by MMPs. During the later stages of the healing process, MMPs are responsible for remodeling the initial scar matrix and maturing the scar. Angiogenesis requires MMP activity. MMPs secreted by vascular endothelial cells degrade the basement membrane, which supports overlying epithelial or endothelial cells, and enable new capillary loops to emerge. (Ladwig, et al. 2002) (Kilpadi, et al. 2006).

A typical wound will heal properly if controlled levels of certain MMPs are produced at specific locations for precise periods of time. One example is seen when the epidermis is regenerated, as MMP-1 is produced by epidermal cells at the leading edge of the migrating sheet. On the other hand, MMP-3 and MMP-9 levels have been shown to decrease during wound healing as shown in a recent study by Kilpadi et al. MMP-9 levels were measured in wound fluid (collected after 1-2 hours of accumulation) using the MMP-9 Biotrak Activity Assay, RPN 2634. Furthermore, it important to recognize that prolonged or elevated levels of certain MMPs are harmful as evidenced in chronic wounds. (Ladwig, et al. 2002) (Kilpadi, et al. 2006).

Increased expression of MMPs and other proteinases is a marker of invasion and metastasis of cancer cells. Initially, MMPIs were used to halt the spreading of cancer, but many problems arose. Cancer trials have been plagued with the inability to identify target inhibition markers and balance target inhibition with efficacy. According to Peterson, a concentration-effect relation based on a biomarker of target inhibition is necessary. This can be generated in phase I studies and serve as a guide for dose-selection and administration schedule for phase II trials. Peterson suggests that validation and selection of the best disease related biomarker(s) in phase II studies would provide a valuable tool to identify patients and monitor efficacy in large-scale phase III trials. (Peterson 2004) (Hu, et al. 2007).

Unfortunately, specific inhibitors for each of the MMP enzymes have not yet been isolated. There is; however, a vast potential market for drugs regulating and targeting MMPs and their inhibitors. It will be necessary to account for: 1) the redundancy of MMPs; 2) the functions of enzyme cascades in balance with natural inhibitors; and 3) activity on non-matrix substrates (e.g., chemokines, growth factors, growth factor receptors, adhesion molecules). (Hu, et al. 2007) (Peterson 2004).

In summary, there are several problems with MMPIs: such as detrimental side effects and patients developing MSS; difficulty managing or determining appropriate dosing; most MMPIs are broad spectrum inhibitors; target selectivity and specificity is mostly unknown; and patient compliance is problematic.

Enzyme-mediated drug delivery. Most of the prior art describes selective release of an active at a site where certain enzymes are uniquely present. For example, U.S. Pat. No. 6,319,518 discloses a colon selective drug delivery composition comprising gelatin and a polysaccharide which is degradable by colonic enzymes; and, optionally, an aldehyde and/or a polyvalent metal ion and/or a colon degradable additional polysaccharide. The colon selective drug delivery system allows lowering of the dose of a drug because the drug can directly act on the colon, thus reducing undesirable and potentially harmful side effects compared with a systemic administration.

U.S. Pat. No. 6,413,494 describes compositions and oral pharmaceutical dosage forms for release of biologically active ingredients in the colon while avoiding or minimizing release into the upper gastrointestinal tract, such as the stomach and small intestine. Due to the lack of digestive enzymes, colon is considered a suitable site for the absorption of various drugs. However, colon drug delivery is hardly achieved in that the oral dosage form should pass through the stomach and small intestine, where many drugs are deactivated by their digestive materials. Ideally, a colon specific drug delivery system is designed such that it remains intact in stomach and small intestine but releases encapsulated drugs only in colon. CSDS system is useful in administering a drug that is an irritant to the upper GI tract, such as non-steroidal anti-inflammatory agents, or drugs that are degraded by gastric juice or an enzyme present in the upper GI tract, such as peptide or protein.

U.S. Pat. No. 6,228,396 and WO 2001/0026807 disclose colonic drug delivery compositions that include a starch capsule provided with a drug and a coating that is broken down by specific enzymes present in the colon. The coating may be a pH sensitive material, a redox sensitive material, or a material broken down by specific enzymes or bacteria present in the colon. The drug to be delivered may be one for local action in the colon or a systemically active drug to be absorbed from the colon.

U.S. Pat. Nos. 5,525,634 and 5,866,619 disclose a drug delivery system including a matrix containing a saccharide-containing polymer that is degraded in the colon by bacterial enzymatic action. According to the invention, the matrix is resistant to chemical and enzymatic degradation in the stomach and small intestine. The matrix is degraded in the colon by bacterial enzymatic action, and the drug is released. The system is useful for targeting drugs to the colon in order to treat colonic disease. The system is also useful for enteric administration of drugs such as proteins and peptides which are otherwise degraded in the stomach and small intestine.

U.S. Pat. No. 5,505,966 discloses a colon selective pharmaceutical composition that includes a matrix core with an active substance and an outer cover layer without the active substance. The core and the cover are selectively degradable by enzymes that are normally present in the colon, wherein the matrix core and the cover layer are comprised of one or more polysaccharides that are selectively degradable by colonic enzymes.

U.S. Pat. No. 5,098,718 discloses a composition for coating of feed additives, such as medicinal products, vitamins and amino acids. The composition is coated with a material that is stable in the rumen, not substantially degraded in the abomasum, and strongly degraded in the small intestines due to the presence of enzymes. The coating includes zein, a hydrophobic substance, an optional water-soluble polymer and organic filler.

Other prior art describes controlled release drug delivery systems with various control mechanisms, but they do not disclose a directed-release systems using a specific target. For example, U.S. Pat. Nos. 6,482,439 and 6,589,563 describe microparticles and nanoparticles including enzymatically degradable polymers that provide drug release at particular sites within the body where the enzyme of interest is present. The particles include a core polymeric matrix in which a drug is dispersed or dissolved and a polymeric shell surrounding the core.

U.S. Pat. No. 6,632,671 discloses nanocapsules and methods of preparing these nanocapsules, including a method of forming a surfactant micelle and dispersing the surfactant micelle into an aqueous composition having a hydrophilic polymer to form a stabilized dispersion of surfactant micelles. The nanocapsule is formed by partitioning a bioactive component within a core of surfactant molecules, and surrounding the surfactant molecules with a biocompatible polymer shell. The nanocapsules may be combined with additional polymeric binders, surfactants, fillers, and other excipients to incorporate the nanocapsules into solid dosage forms such as granules, tablets, pellets, films or coating for use in enhanced bioactive component delivery. In this way, design of the dissolution profile, control of the particle size, and cellular uptake remains at the level of the nanocapsule.

U.S. Pat. No. 4,774,091 discloses a solid sustained-release preparation which includes an active ingredient and a pharmaceutically acceptable biodegradable carrier. Suitable biodegradable carriers include collagen, gelatin, albumin, and the like. The sustained-release preparation can be administered to the body or implanted into the body by injection or an injection-like method and can release the active ingredient at an effective level for a long period of time when administered. This is merely an example of drug delivery vehicles that include collagen. There are additional references that disclose the use of collagen for drug delivery, but may not specifically identify the enzymatic degradation property of the collagen.

There has yet to be an invention that takes advantage of enzyme-mediated drug delivery related to areas that include MMPs in a concentration-specific manner, i.e., wherein vehicle may be degraded by a basal level of MMP or selectively with a specific (elevated) concentration of MMP).

### Example 1

Development of hydrogel based MMP responsive formulation for controlled release of loaded bio-molecules.

Introduction: A hydrogel based formulations, which can be topically applied to skin, to protect against UV damage was developed. It has been shown that exposure to UV particularly UV-B leads to up-regulation of MMP-9. Activation of MMP-9 has been associated with photo induced ageing effects.

In a phase-1 of this study, a gelatin based hydrogel was used to model and demonstrate degradation of MMP responsive hydrogels. Gelatin was selected as a model system in this study. The hydrogels are formed by cohesive interaction of polymeric materials. These interactions can be further strengthened by chemical crosslinking of polymers. Crosslinking of polymers provides increase the mechanical strength as well as control in release characteristics of these hydrogels. The major advantage of hydrogels is efficient delivery of large pay loads of bioactives compounds.

In this application, gelatin based hydrogels were crosslinked to improve retention of bio-active molecules. Further these bio-active molecules may be encapsulated within microspheres which are dispersed in the crosslinked hydrogel. In many applications, this encapsulation approach may be desired to improve the solubility and bio-availability of hydrophobic based bioactive compounds, e.g., retinol A. In this specific application, hydrophobic dye molecules are encapsulated in PLGA microspheres and loaded in a gelatin crosslinked hydrogel. Hydrogels in this study are cross-linked using glutaraldehyde. Since the mechanical characteristics of hydrogels have a significant effect on the controlled release characteristics, we have also investigated hydrogels with different bloom strengths of gelatin for control release of bio-actives.

Summary of Results: Gelatin Hydrogels formulations. Use of polymeric microparticles with a crosslinked hydrogel network provides further control on release of tracer dye molecules. Based on experimental testing it was found that polymeric nanoparticles are required to control release over 12-48 hour period. It was found that Bloom 300 gelatin based hydrogels provide a better release control as compared with bloom 100 gelatin. Varying % of gelatin from 2-10% did not significantly affect the release rates, especially without crosslinking.

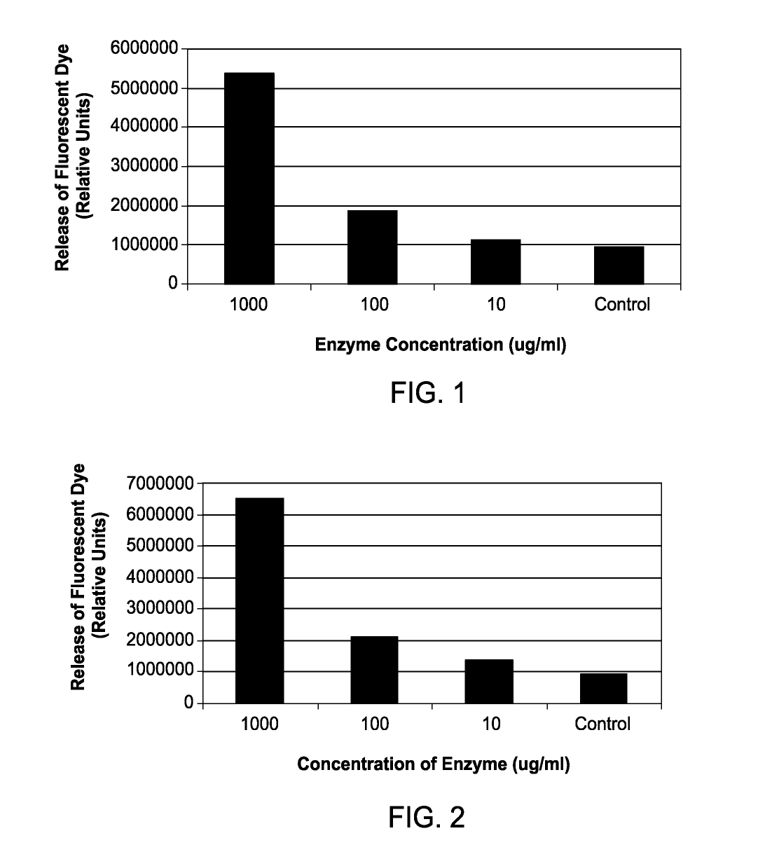

Initial testing with enzymatic treatment show ˜5 fold higher release of tracer dye from gelatin hydrogels as compared to control, in a concentration dependent manner. These gelatin hydrogels were loaded with PLGA microparticles with tracer dye encapsulated within polymeric microparticles. The difference in total release was observed after overnight incubation at 37° C. (FIGS. 1 and 2).

FIG. 1 is a graph that shows the release of fluorescence dye from PLGA microspheres in a gelatin hydrogel 75:25 (bloom 300) treated with MMP-9 enzymes at 37° C. FIG. 2 is a graph that shows the release of fluorescence dye from PLGA microspheres in a gelatin hydrogel 50:50 (bloom 300) treated with MMP-9 enzymes at 37° C.

### Example 2

Development of microparticles based MMP responsive formulation for controlled release of Bioactive compounds.

In a complementary formulation approach, we have developed microparticles based formulation for MMP responsive controlled release of bioactive compounds. The potential advantage of microparticles based formulations is the ability to add bioactive molecules to diverse topical formulations. In addition, microparticles due to thin shell thickness provide a rapid release mechanism for bio-active compounds.

In this study, the aim was to develop 10-20 micron sized microparticles in which the shell material can be degraded by active MMP enzymes. As a model system, we prepared gelatin microspheres loaded with tracer dye dissolved in canola oil.

Summary of Results: Gelatin Microparticles formulations. Prepared gelatin microspheres loaded with tracer dye dissolved in canola oil. Size of gelatin microspheres was examined using microscopy. The microspheres are ˜10 microns in diameter. Optimized the crosslinking procedure using 0.5% glutaraldehyde to reduce aggregation. Measured changes in microparticles structure following enzymatic treatment using optical imaging. Human MMP-9 is effective in degradation of gelatin in microcapsules. Conc. of 1 μg/ml is effective for in-vitro degradation in overnight incubations. At low concentration of 1 ng/ml, longer incubation time period is required. With 10 units of bacterial derived MMP Enzymes/ml most of the shell degradation was achieved in ˜3 hours on incubation. With bacterial derived enzyme concentration of 1 units/ml and 0.1 units/ml, samples required overnight incubation to achieve shell degradation. Measured release kinetics to quantify the total release over an incubation period.

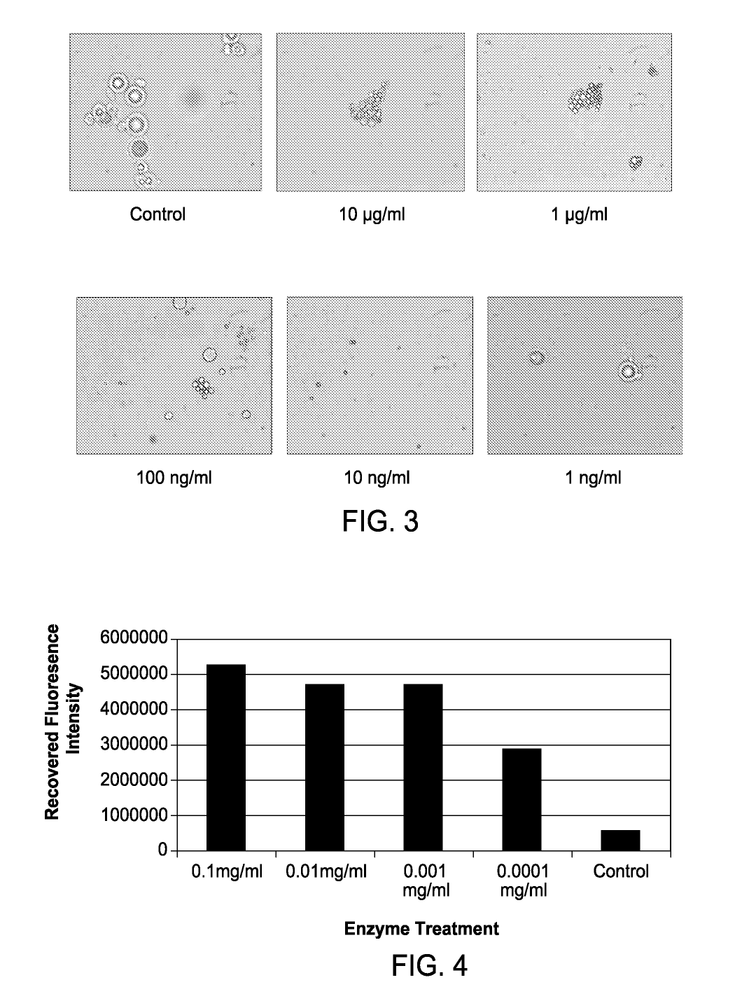

FIG. 3 is a micrograph that shows the degradation of gelatin shell in microcapsules treated with varying concentration of MMP-9 enzymes for 12 hours at 37° C. Degradation of shells leads to release of oil phase, which shows aggregation in aqueous buffer.

FIG. 4 is a graph that shows the release of fluorescence dye after treatment of gelatin microcapsules with varying concentration of MMP-9 enzymes. The results show ˜15 fold release with enzyme concentration of 1 ug/ml.

### Example 3

MMP-1 responsive microparticles. In this example, the formulation and evaluation of MMP-1 responsive microparticles for controlled release applications was determined. MMP-1 enzymes are also known as collagenase enzymes. These enzymes have a critical role in, e.g., UV-triggered photo-ageing of skin, in wound beads, chronic inflammatory diseases. Thus, developing a controlled release formulation of MMP-1 microparticles can have a significant impact across multiple applications in cosmoceutical and biomedical fields. This example demonstrates the development and characterization of MMP-1 responsive microparticle formulations. The formulations in this example were developed using collagen-I as a model substrate for MMP-1 activity. Various active ingredients (e.g. dye molecules, antibiotic molecules) were encapsulated in MMP-1 responsive formulation. In one example, the polymeric protein matrix is a collagen microparticle formulated using sonication of collagen in presence of a small molecular weight surfactant (e.g., sodium cholate), with an active agent and a protein crosslinker. For testing purposes and in some final applications, the microparticles may also include at least one of a dye, an excipient, a stabilizer, a buffering agent, an anti-oxidant, a salt, one or more inert agents and/or additional active agents.

Material and Methods: Formation of Collagen Microparticles. Collagen microparticles in this study were formulated using sonication of Collagen in presence of sodium cholate (small molecular weight surfactant). The formulation was developed using 5 ml of collagen solution 0.75% Collagen, 5 mL of 0.75% sodium cholate mixed with 50 ul of dyes or concentrated solution of vancomycin and 50 ul 0.1% 1-Ethyl-3-[3-dimethylaminopropyl]carbodiimide hydrochloride (EDC) crosslinker. The microspheres formed in this process were washed and centrifuged. The microspheres were maintained at 4° C. throughout various processing steps. The collagen or other protein percent in solution may be 0.1, 0.5, 0.75, 1.0, 1.5, 2, 2.5, 5, 7.5 or 10%. The percent of the small molecular weight surfactant may also be 0.1, 0.5, 0.75, 1.0, 1.5, 2, 2.5, 5, 7.5 or 10%. Likewise, the crosslinker can be 0.1, 0.5, 0.75, 1.0, 1.5, 2, 2.5, 5, 7.5 or 10% in solution when added to the mixture of the protein, active agent and surfactant.

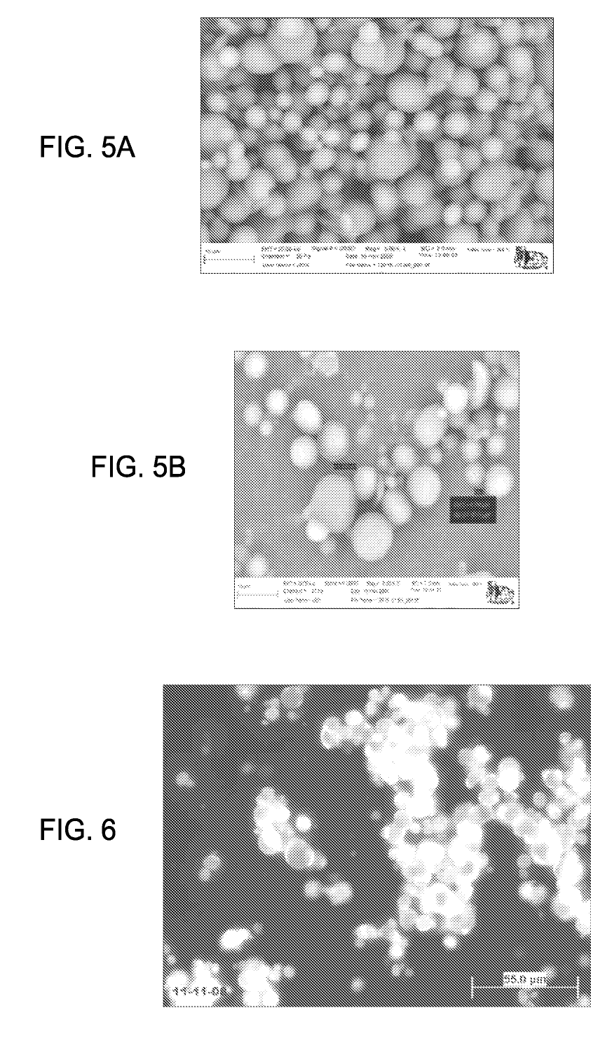

Results: Structural Analysis of Microparticles: To characterize the size and uniformity of collagen microparticles, microparticles were imaged using SEM. The results of SEM imaging are shown in FIGS. 5A and 5B. The results show collagen microparticles are ˜10-20 microns in diameter. The results highlight some degree of polydispersity in the sample. This polydispersity in size of microparticles may be due to potential limitations of lack of uniformity during the sonication process. To avoid denaturation of collagen during homogenization, we limited the time duration of sonication to 30 seconds-1 minute interval, however, times for sonication include 10, 20, 40, 60, 90, 120, 180, 240, or more seconds; or 3, 4, 5, 6, 7, 8, 9, 10, 15, 20, 30, 45 or 60 minutes. A short interval of sonication leads to polydispersity in microparticle formulation.

Distribution of Collagen in Microparticles: The distribution of collagen in microparticles is important for the development of controlled release of an encapsulant material by degradation of collagen by MMP-1 enzyme. To demonstrate that the collagen is present on the outside surface of microparticles; the microparticles were fluorescently labeled the with a dye molecule. The dye molecules selected for this example react specifically with protein molecules, thus, presence of fluorescent dye in a microparticle provides information regarding spatial distribution of collagen in the microparticles. The results of fluorescent imaging are shown in FIG. 6. The results highlight that collagen microparticles have a uniform distribution of collagen both inside and at the surface of microparticles. The intensity of the fluorescence labeling is higher at the surface and decreases towards the center of a microparticle. The drop in intensity may be the result of the 3-D structure of collagen particles, which limits the effective excitation of interior section of microparticles.

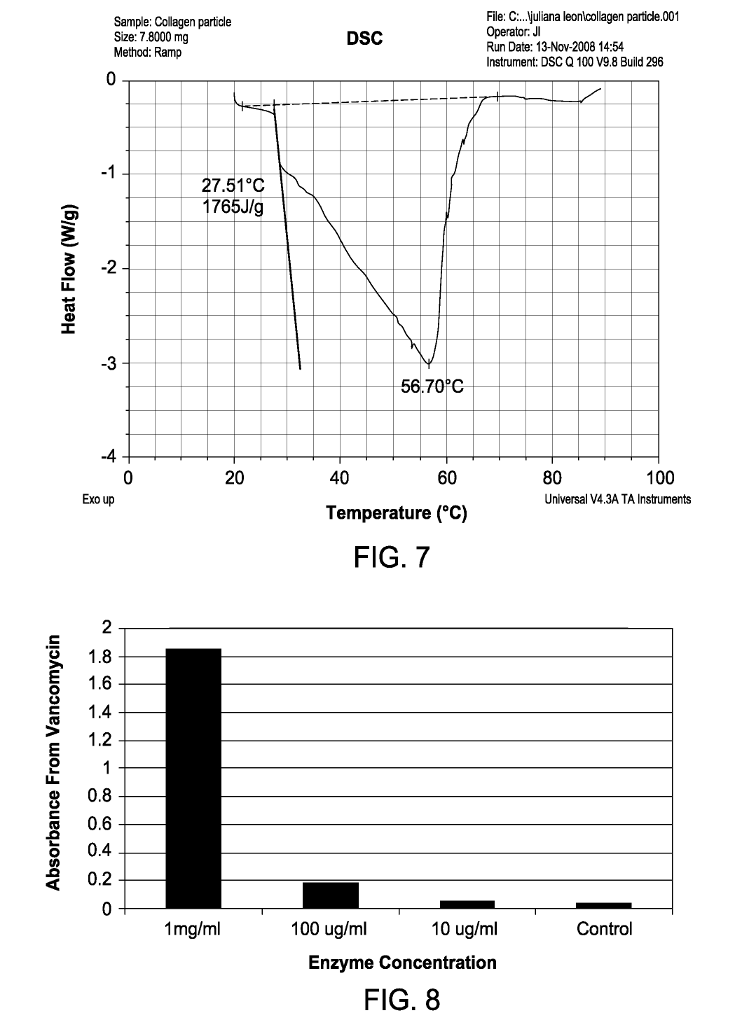

Stability of Collagen in Microparticles: Collagen stability in microparticles is an important factor for development of MMP-1 responsive formulations. This is an important factor because of the structural specificity of MMP-1 enzyme for the collagen substrate. To ensure structural stability of the collagen during formulation, dynamic scanning calorimetric analysis (DSC) was conducted. The DSC results provide a thermodynamic analysis of denaturation of collagen with elevation of temperature. If the collagen retains its native structure during process, it is expected that the DSC results will show a phase transition upon denaturation of collagen fibers. Similarly, if the collagen is denatured during processing, the phase transition will not occur with an increase in temperature. DSC analysis has used to characterize structural analysis of collagen solutions. A representative plot of DSC study is shown in FIG. 7. Results show that the collagen fibers in microparticles maintain their native structure and are not denatured during the process. This result validates that the process outlined in the methods section of this report can be used to develop microparticles, while maintaining the native structure of collagen.

Encapsulation and Controlled Release of Bio-active Ingredients upon Treatment with MMP-1: The next step in development of MMP-1 responsive microparticles was to encapsulate active ingredients and demonstrate controlled release of active ingredients with MMP-1 activity. The results of encapsulation and controlled release of vancomycin encapsulated in collagen microparticles is shown in FIG. 8. These results clearly highlight that vancomycin can be stably encapsulated in collagen microparticles and can be released upon incubation with MMP-1 enzyme. In this study, vancomycin concentration was measured using a UV-Vis analysis.

### Example 4

Development and testing of MMP-9 responsive microparticles. This example shows an evaluation of stability of gelatin coacervate microparticles.

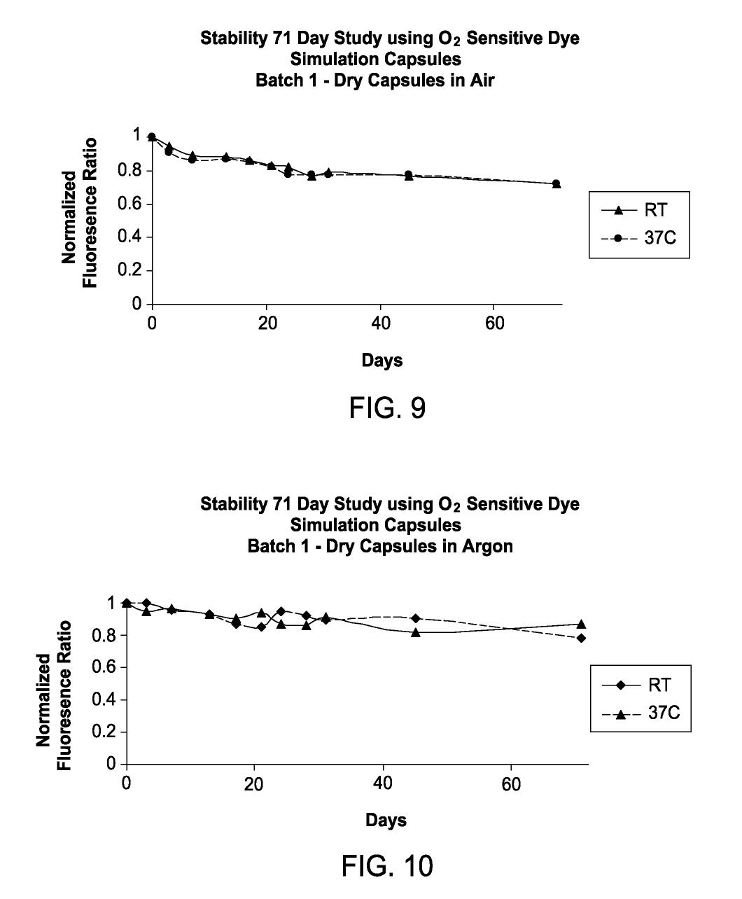

Oxidative Stability of Gelatin Coacervates (MMP-9 responsive microparticles): Structural stability of gelatin coacervates. In this example, the oxidative stability of gelatin coacervates was determined. Oxidative stability is important for variety of cosmetic and cosmoceutical applications. The results of the study are summarized in FIG. 9-11. The results highlight that diffusion of oxygen in dry capsules in air is slow (FIG. 9). Over 71 days of evaluation, the oxygen sensitive dye retained its fluorescence at 76% of its starting level. The results also indicate that temperature of storage does not affect the permeation of oxygen across gelatin shell. These data indicate that gelatin coacervate shell in dry conditions provide excellent oxygen permeability barrier.

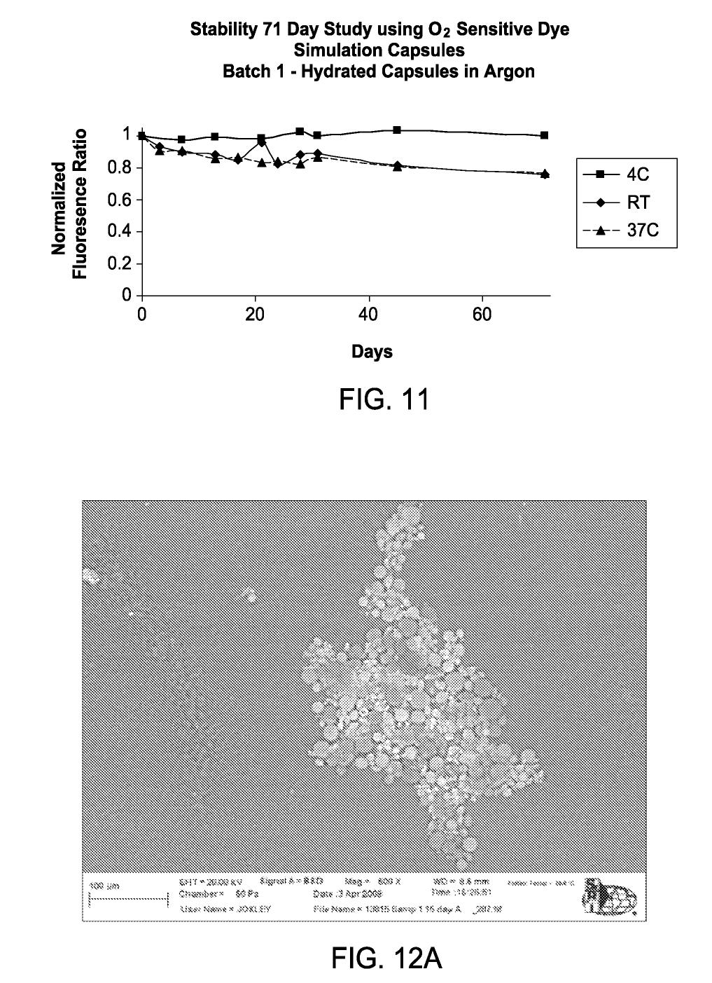

A similar study was also conducted to evaluate stability of gelatin coacervates stored in argon and also in solution purged with argon. The results of these studies are shown in FIGS. 10 and 11, respectively. Comparison of results in FIGS. 9 and 10 indicates that although storage of gelatin coacervates in argon provides a more stable environment but at the same time the difference in oxygen permeation during storage in air and argon is not drastically different. These results indicate that gelatin coacervates are stable in air over an extended storage. Further both results indicate that storage temperature has no significant effect on diffusion of oxygen under dry storage conditions.

FIG. 11 shows the stability analysis of gelatin coacervates stored in aqueous environment purged with argon. The results show that in an aqueous solution the temperature has a significant effect on diffusion of oxygen across gelatin shell. Further comparison of these results with storage in dry conditions indicates that dry conditions provide better stability for oxidation sensitive products as compared to wet conditions.

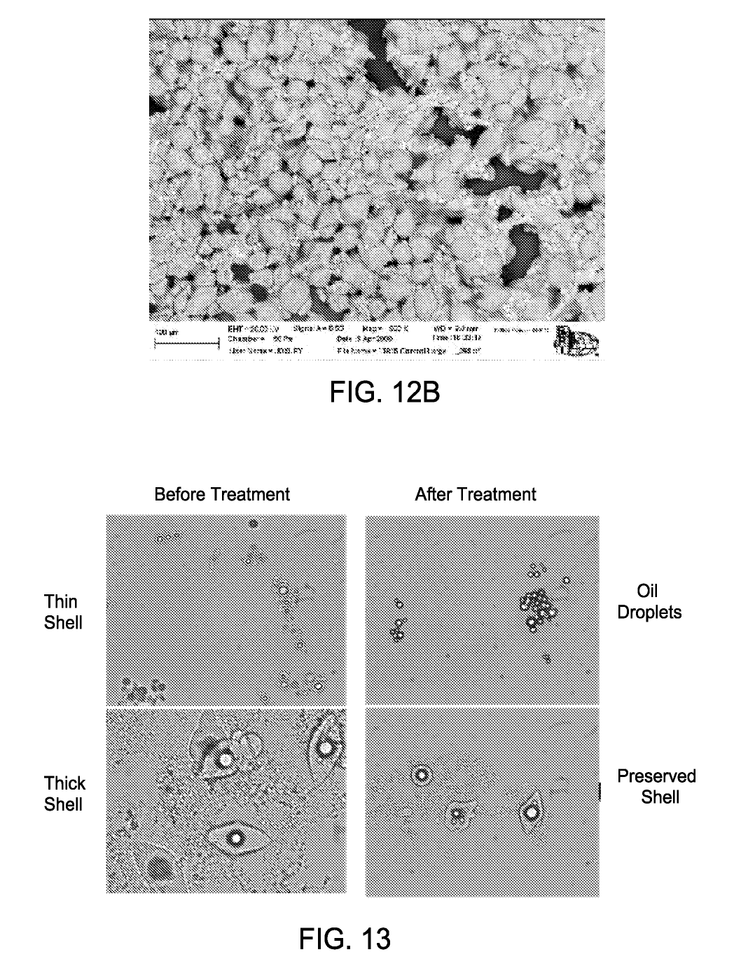

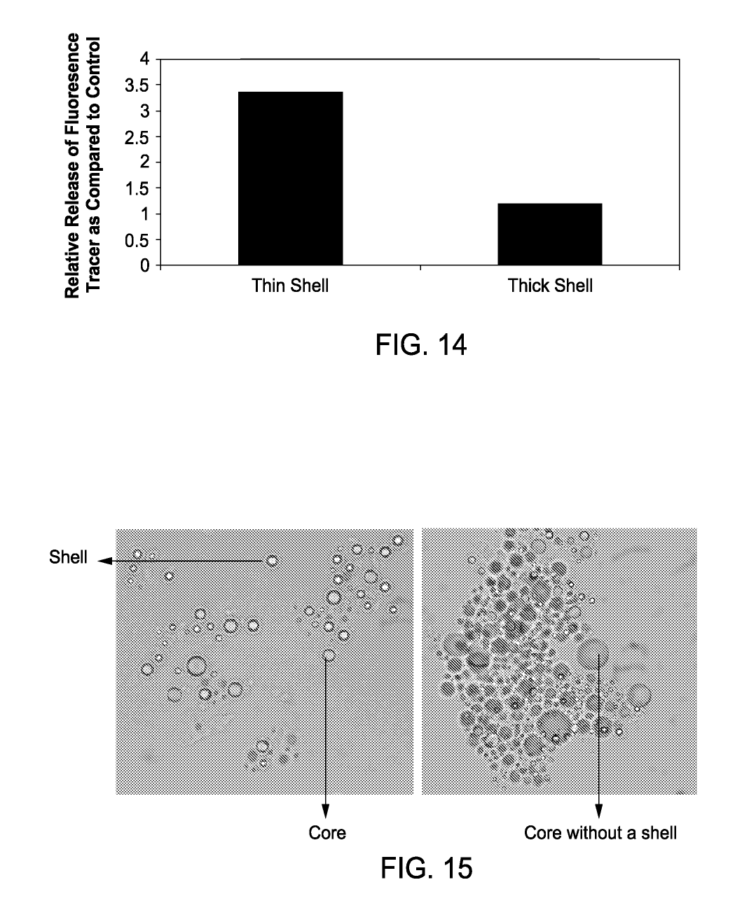

FIGS. 12A and 12B show control the kinetics of release of bioactive compounds with a thin shell (12A) and a thick shell (12B). FIG. 13 shows the release kinetics of thin vs. thick shell. FIG. 14 is a graph that compares the release kinetics of thin vs. thick shell wherein the engineered shell thickness reduces the rate of release from core-shell microparticles by ˜3 fold.

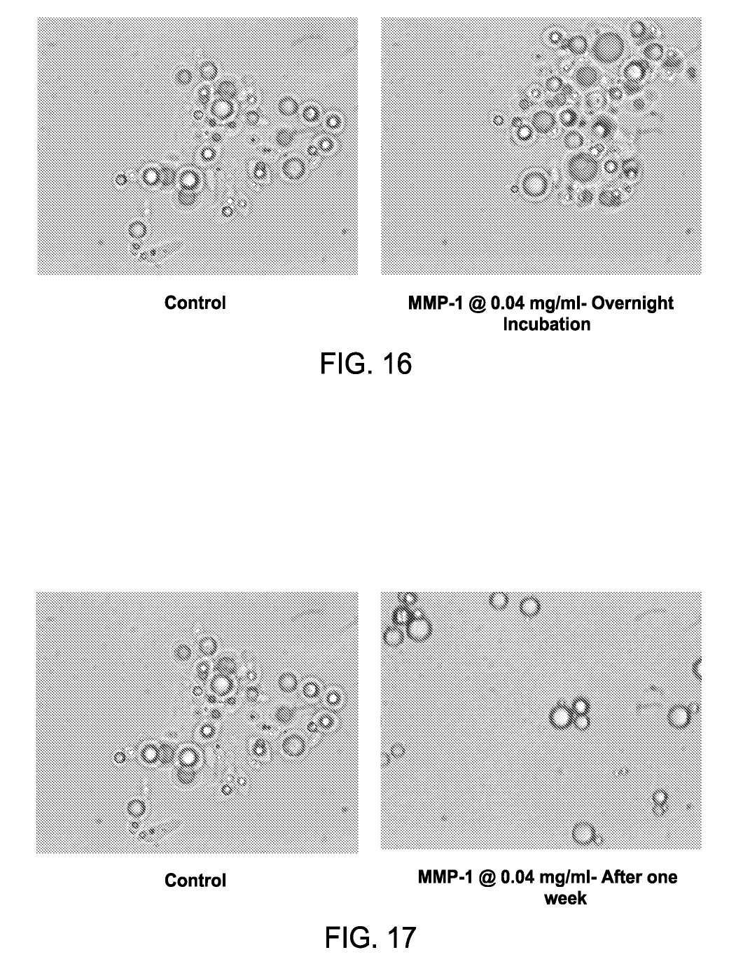

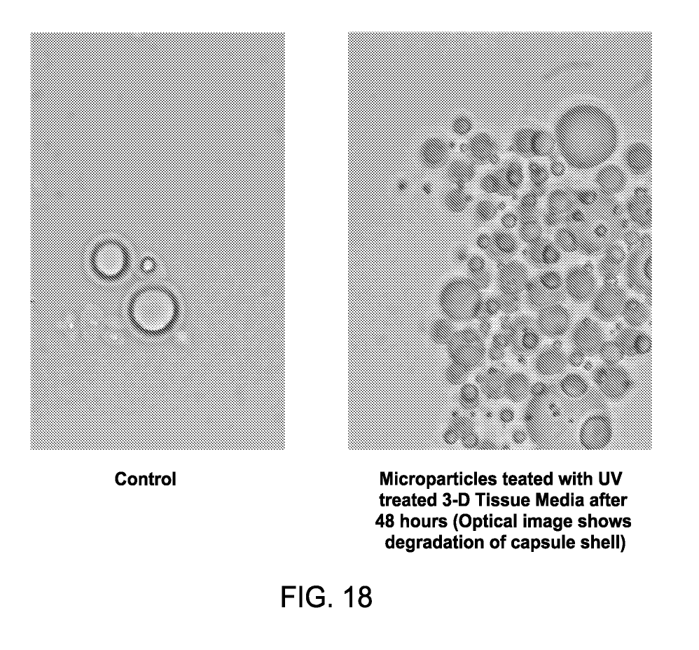

FIG. 15 shows the degradation of the shell using activated PBS-buffer 20 minutes after UV exposure, the particles were incubated overnight with the activated PBS sample. FIG. 16 shows the specificity of the protease against the shell, in this example, a lack of MMP-1 activity after 24 hours to gelatin capsules degraded by MMP-9. FIG. 17 shows the specificity of the protease against the shell, in this example, a lack of MMP-1 activity after 1 week to gelatin capsules degraded by MMP-9. Studies shows high specificity of MMP-9 and MMP-1 enzymes; very slow degradation as visualized. Overnight incubation with a large concentration −40 μg/ml of MMP-1, which is significantly larger than the physiologically relevant concentration (˜10-200 ng/ml) demonstrates negligible degradation of particles. FIG. 18 shows microparticles treated with UV treated 3-D tissue media. MMP-9 is not a dominant MMP produced in response to UV treatment as compared to MMP-1 (MMP-9 is 8-10 fold lower as compared to MMP-1), however the concentration of MMP-9 was sufficient to cause disruption of the capsules after this period of time.

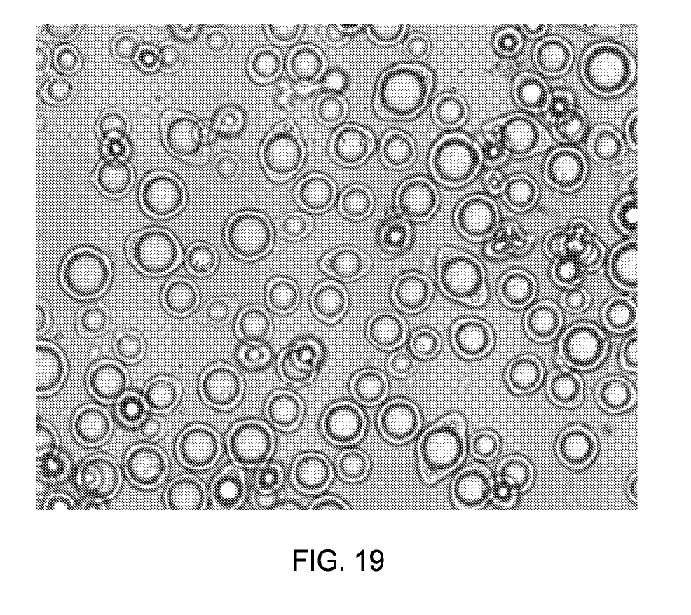

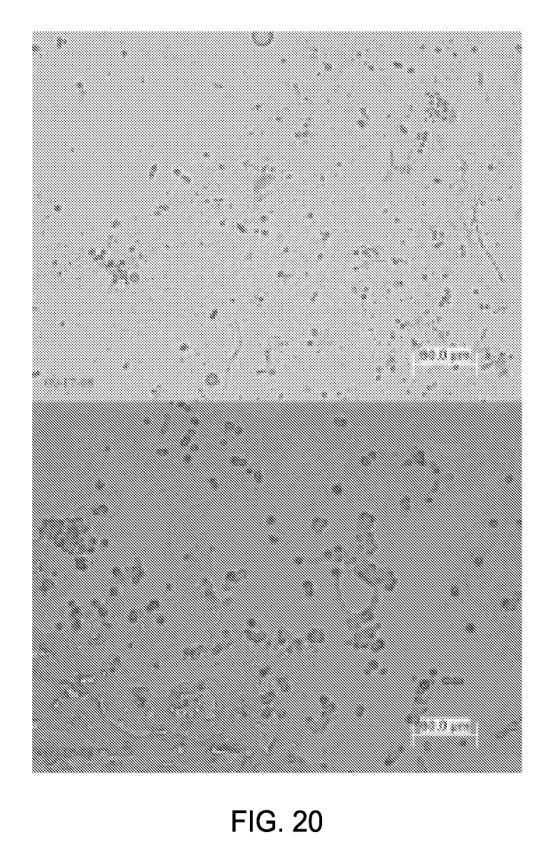

FIG. 19 shows gelatin microcapsules encapsulating benzoyl peroxide. FIG. 20 shows the encapsulation (top) and release (bottom) of Trypan Blue dye in collagen. These pictures of the 10 ml collagen microspheres are shown here. What is interesting to note is surrounding them are collagen fibers, which seem to form sheets. FIG. 20 (top): This is a 10× image created on a Lieca Florescent Microscope with a color camera. FIG. 20 (bottom): This is a 20× image created on a Leica Florescent Microscope of the dyed particles surrounded by collagen.

Generally, all technical terms or phrases appearing herein are used as one skilled in the art would understand to be their ordinary meaning. It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A method of controlling the release of an active agent with a proteinase comprising:
encapsulating one or more active agents with a crosslinked or uncrosslinked matrix, the matrix being cleavable by the proteinase; wherein exposure of the matrix to one or more proteinases causes the cleavage of the matrix and the release of the active agents.

2. The method of claim 1, wherein the matrix is formulated such that cleavage and degradation of the matrix occurs in a proteinase enzyme concentration specific manner.

3. The method of claim 2, wherein the concentration of proteinase comprises a threshold concentration below which agents present within the matrix are not released.

4. The method of claim 3, wherein the threshold concentration comprises a level that is higher than a basal level of proteinase enzyme activity in normal tissue.

5. The method of claim 3, wherein the threshold concentration comprises a level that is higher than a basal level of proteinase enzyme activity in normal tissue over a specific time period.

6. The method of claim 1, wherein the proteinase is a matrix metalloproteinase (MMP).

7. The method of claim 1, wherein the proteinase is a matrix metalloproteinase 1, MMP2, MMP8, MMP9 and Gelatinase.

8. A tunable system for delivery of agents comprising a matrix comprising an active agent and a substrate for a proteinase enzyme and which, upon exposure to the proteinase is degraded thereby triggering the release of the active agent.

9. The system of claim 8, wherein the matrix is crosslinked.

10. The system of claim 8, wherein the time and rate of delivery is tuned by adjusting the amount of substrate, the amount of crosslinking, the type of crosslinking and combinations thereof.

11. A method of making a composition that controls the release of an active agent upon exposure to a metalloproteinase comprising:
encapsulating one or more active agents with a polypeptide-matrix encapsulant comprising cleavable polypeptides that are susceptible to cleavage by the metalloproteinase and a polymer;
wherein exposure of the encapsulant to one or more metalloproteinases triggers the cleavage of the polypeptide and the release of the active agents.

12. The method of claim 11, wherein the proteinase is a matrix metalloproteinase 1, MMP2, MMP8, MMP9 and Gelatinase.

13. The method of claim 11, wherein the cleavable polypeptides comprise at least one of collagen (Types I to XIV), elastin, gelatin, fibronectin, laminin and basal membrane proteins.

14. The method of claim 11, wherein the composition is formed into a thin film coating adapted for topical, parenteral, oral, buccal or subcutaneous administration.

15. The method of claim 11, wherein the polymer-polypeptide matrix is formed by sonicating collagen in solution in presence of sodium cholate and a crosslinker.

16. The method of claim 11, wherein the composition is formulated into a product for pharmaceutical, cosmetic, cosmaceutical, dermal or wound care use.

17. The method of claim 11, wherein the composition is formulated as nano, micro or mini capsules.

18. The method of claim 11, wherein the composition is formulated as a hydrogel.

19. The method of claim 11, wherein the composition is formulated as nano, micro or mini capsules each comprising a different release profile that are combined into a multi-release formulation.

20. The method of claim 11, wherein the composition is used to coat a device.

21. The method of claim 11, wherein the composition is integrated into an active agent delivery device that does not degrade until exposed to a metalloproteinase.

22. The method of claim 11, wherein the composition is incorporated into an implant, a bandage or a dressing.

23. The method of claim 11, wherein the composition is formed into multiple particles with different drugs and the polypeptides are a substrate for the same metalloproteinases.

24. The method of claim 11, wherein the composition is formed into multiple particles with different drugs and the polypeptides are a substrate for different metalloproteinases.

25. The method of claim 11, wherein the composition comprises multiple layers that are susceptible to a first and a second metalloproteinases.

26. The method of claim 11, wherein the composition comprises multiple layers each loaded with a different active agent and wherein each layer is susceptible to different metalloproteinases.

27. The method of claim 11, wherein the composition further comprises dyes, tracers, labels, contrast agents, or other detection agents.

28. The method of claim 11, wherein the composition is formulated into at least one bead, each bead comprising a different amount of polymer-polypeptide matrix material, degree of crosslinking, nature of crosslinking or combinations thereof, where upon exposure to a delivery site, the presence of a specific and constant concentration of metalloprotease at the delivery site differentially degrades the beads and triggers a unique release profile, selected from at least one of a zero order release, pulsatile release, delayed release, and increasing release.

29. The method of claim 11, wherein the composition is formulated into a plurality of beads, each bead comprising a different polymer-polypeptide matrix matched with a particular detection agent, where upon exposure to a delivery site, the presence or absence of a metalloprotease at the delivery site is detected by the detection agent released from the beads.

30. The method of claim 11, wherein the composition is formulated into at least a portion of an indicator placed in a wound, derma, cosmetic/cosmoceutical, oral, implants, tumors (metastasis) or a device.

31. The method of claim 11, wherein the composition is formulated into a plurality of beads, each bead comprising a different polymer-polypeptide matrix matched with a particular detection agent, where upon exposure to a delivery site, the presence or absence of a metalloproteinases at the delivery site is detected by the detection agent released from the beads on a cardiac device.

32. The method of claim 11, wherein the polymer-polypeptide matrix further comprises one or more tissue inhibitors of metalloproteinases.

33. The method of claim 11, wherein the polymer-polypeptide matrix is a biopolymer.

34. A composition for the controlled release of an active agent comprising:
a matrix comprising a polymeric portion crosslinked by a polypeptide that is susceptible to cleavage by a metalloproteinase, wherein erosion of the matrix by exposure to a metalloproteinase that is specific for the protein causes the release of one or more active agents encapsulated by the matrix.

35. The composition of claim 34, wherein the matrix comprises a gelatin coacervate.

36. The composition of claim 34, wherein the composition comprises a protein microparticle, a small molecular weight surfactant, one or more active agents and a protein crosslinker.

37. The composition of claim 34, wherein the composition comprises a collagen microparticle, sodium cholate, one or more active agents and a collagen crosslinker.

38. The composition of claim 34, wherein the composition is formulated into microparticles that further comprise at least one of a dye, an excipient, a stabilizer, a buffering agent, an anti-oxidant, a salt, or one or more inert agents.

39. The composition of claim 34, wherein the proteinase is a matrix metalloproteinase 1, MMP2, MMP8, MMP9 and Gelatinase.

40. A composition for the controlled release of an active agent made by a method comprising:
encapsulating one or more active agents with a polymer-polypeptide matrix, wherein the polypeptide are susceptible to cleavage by metalloproteinases; and
exposing the polymer-polypeptide matrix to one or more metalloproteinases, wherein the cleavage of the polypeptide causes the release of the encapsulated active agents.

41. The composition of claim 40, wherein the polymer-polypeptide matrix further comprises one or more protease inhibitors.
